(19) United States
(12) Patent Application Publication
(10) Pub. No.: US 2006/0025818 A1
(43) Pub. Date: Feb. 2, 2006

(54) Strong, flexible suture material

(76) Inventor: Patrick J. Ferguson, Portland, OR (US)

Correspondence Address: GLENN C. BROWN, PC, 777 NW WALL STREET, SUITE 308, BEND, OR, 97701, US

(21) Appl. No.: 10/989,647

(22) Filed: Nov. 15, 2004

Related U.S. Application Data
(60) Provisional application No. 60/592,808, filed on Jul. 29, 2004.

(51) Int. Cl. A61L 17/00

(52) U.S. Cl. 606/228

(57) ABSTRACT

The present invention provides for a suture comprising a flexible foraminous cover made of a polymeric material, and a core within the cover made of a cushioning material. The cover material is preferably formed of uhmw polyethylene, nylon or polypropylene. The cushioning material is preferably polyester, PTFE or nylon. Preferably, the core is twisted at about 1.5 turns per inch and comprises about 22% of the suture.

This application is a Continuation-in-Part of provisional application 60/592,808, filed on Jul. 29, 2004.

### BACKGROUND OF THE INVENTION

1. Technical Field

This invention relates to surgical sutures, and in particular to a composite suture comprised of an inner core and an outer sleeve.

2. Field of the Invention

Most all surgical procedures requiring incisions are concluded by closing the incision with stitches of a suture material. Sutures were originally manufactured from animal or plant derived materials. As synthetic, polymeric materials have been developed, many of those materials have been used to make sutures. Sutures come in a wide variety of sizes, and are made from both bio-absorbable and non-absorbable materials. In almost every instance where sutures are required it is desirable to use the suture having the best combination of high tensile strength, small diameter, flexibility, knot strength and knot security.

Sutures are made in a variety of basic configurations, including monofilament, braided, stranded, and sutures having a core material encased within an outer cover. Some examples are described in U.S. Pat. Nos. 6,716,234, 5,318,575, 5,314,446,4,700,704, and in U.S. Ser. No. US2003/0050666 A1.

### BRIEF SUMMARY OF THE INVENTION

This invention provides a further improvement on existing suture materials by providing a suture that embodies high tensile strength, high knot strength and good knot security, and which at the same time is very flexible and has an unexpectedly high strength relative to its relatively small diameter. A preferred embodiment of a suture according the present invention generally includes a core comprising polyester fibers which are twisted at about 1.5 turns per inch. The core is encased in a sleeve comprising braided, long-chain, high molecular weight polyethylene terephthalate threads.

### BRIEF DESCRIPTION OF THE DRAWINGS

The nature, principle and utility of the present invention will be clearly understood from the following detailed description when read in conjunction with the accompanying drawings, wherein:

FIG. 1 is a cross-sectional view of the suture of the present invention.

FIG. 2 is an enlarged view of a segment of the suture of the present invention.

FIG. 3 is a perspective view of the core of the suture of FIGS. 1 and 2 according to a first embodiment.

FIG. 4 is a perspective view of the core of the suture of FIGS. 1 and 2 according to a second embodiment.

The drawings are for illustrative purposes only and are not drawn to scale. In the drawings, the same numbers are used for the same part or portion throughout the drawings.

### DETAILED DESCRIPTION OF THE INVENTION

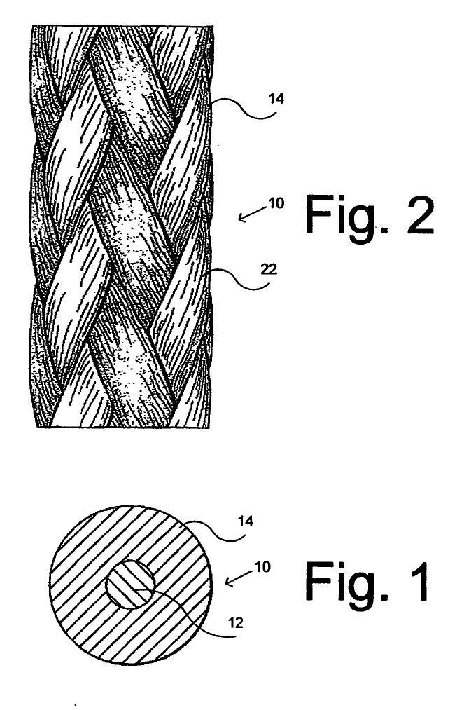

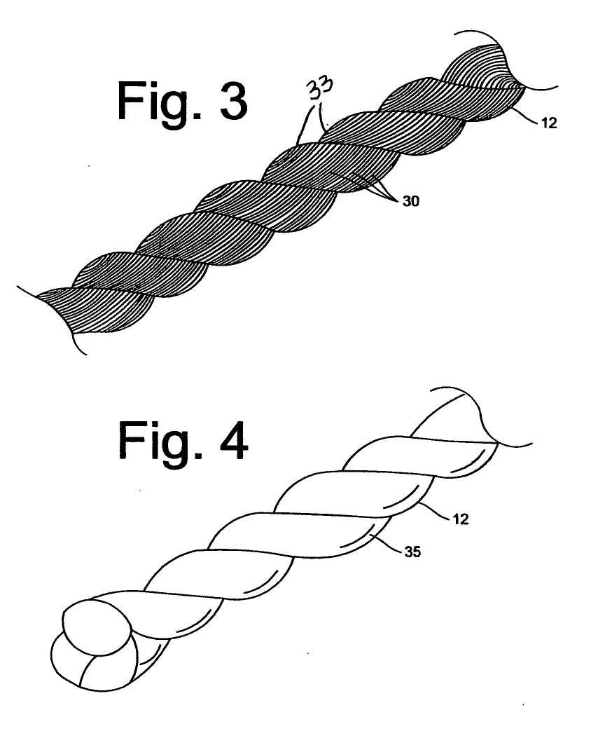

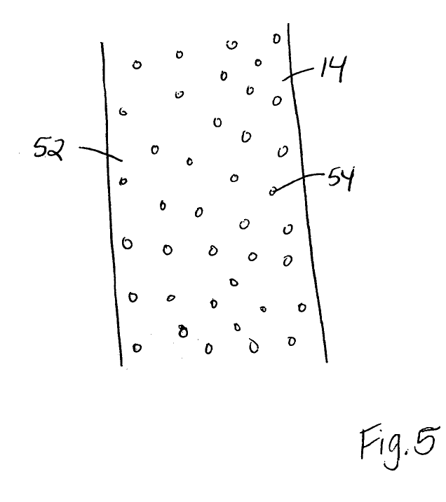

Referring now to FIGS. 1-4, the invention will be described in greater detail. A suture 10 according to a preferred embodiment of the invention includes a core 12 and an outer sleeve 14. Core 12 comprises a plurality of threads that are twisted together at preferably between 0.5 and 2.5 turns per inch, and most preferably at about 1.5 turns per inch. Referring to FIGS. 3 and 4, core 12 is preferably comprised of twisted, long-chain polyester fibers. As an alternative to polyester, the core could be made of twisted threads of PTFE, nylon, or other materials with the yield and cushioning properties discussed below.

In certain preferred embodiments the core material preferably comprises between about 10% and 30% of the total material in suture 10, more preferably between about 15% and 25%, and in one preferred embodiment about 22% of suture 10. In other embodiments of the invention the core material could comprise a greater or lesser proportion of the material.

FIG. 3 shows one preferred embodiment that includes multiple fine polyester strands 30. Strands 30 are twisted together to form threads 33, which are in turn twisted together to form core 12. FIG. 4 shows a second preferred embodiment in which a multiple single strands 35 are twisted together to form core 12.

In the preferred embodiments core 12 is sheathed in a woven or braided cover 14. Cover 14 is formed from multiple threads of strong, abrasion resistant material that is also flexible owing to its woven or braided construction. In one preferred embodiment the cover is formed of braided, ultra high molecular weight (uhmw) polyethylene terephthalate fibers 22 in an elongate, hollow configuration. In some preferred embodiment the umhw polyethylene terephthalate fibers 22 are selected and braided to provide a cover 14 with between about 50 and 60 picks per inch, and in one preferred embodiment 52 picks per inch. In other embodiments the cover could be formed from other high strength polymeric materials such as nylon or polypropylene.

In still other embodiments the cover material 14 could be formed from a non-woven, non-braided material 52 that includes multiple openings 54 shown in FIG. 5, similar in construction to “expanded” polymeric or metallic materials which are formed of a unitary material having openings formed therein. In this embodiment, the hollow, flexible cover is made of a foraminous, abrasion resistant polymeric material. The openings 54 can be rounded as shown in FIG. 5 or polygonal (not shown), and a cover material formed in that configuration embodies the combined qualities of strength and flexibility and in that way is similar to a braided or woven cover. The advantage of this configuration is that it could be formed in a single extrusion step.

In the structure and materials of a suture according to the invention, the braided or woven cover 14 provides strength, abrasion resistance and flexibility. The polyester core 12 provides additional strength, and at the same time it acts as a cushion in the suture structure. The polyester core supports the braided cover in a normally round shape during insertion of the suture into the needle eye. In addition, the core permits a cushioning deformation of the cross-sectional shape of the cover when the suture is in a bent configuration. As the suture is knotted, the structure allows the suture to partially collapse and tightly cinch to provide enhanced knot strength, while at the same time preventing the braided cover from completely collapsing and kinking, which can weaken the suture. The cushioning core provides this protection by allowing the suture to easily bend, but at the same time limiting the radius of curvature of the bent suture to less than the yield point of the cover material. Another benefit of the polyester core is that it does permit some give in the suture to accommodate stresses in the tissue that normally occur as the patient moves.

In one example of a suture according to the preferred embodiments described above, a suture was manufactured having a hollow, braided, uhmw polyethylene terephthalate cover having 52 picks per inch and an overall diameter of 0.026″. The cover surrounded a polyester core containing 22% of the total material. The suture exhibited excellent flexibility, an unexpectedly high breaking strength of about 108 pounds, and a relatively low elongation of 5%. The excellent flexibility results in unexpectedly good knot compression, while at the same time the suture exhibited improved knot strength over other sutures. For example, the knot strengths of the Ethibond™ size #2 and the leading polyblend size #2 sutures are about 12.8 pounds and 27.2 pounds, respectively. The knot strength of the suture of the preferred embodiment is about 46.3 pounds. This unexpectedly high knot strength provides enhanced knot security, which is important when closing incisions and wounds.

The core and cover of the preferred embodiment result in a suture with an unexpectedly high strength relative to its relatively small diameter. The suture of the present invention is about three times stronger than polyester of the same size and about 15 times stronger than steel on a weighted basis.

In another aspect of the invention the relatively soft, stranded polyester core combined with the braided cover provides a soft, compliant suture that forms tight, compact knots, while the uhmw polyethylene terephthalate cover provides good abrasion resistance in a form that is also very flexible and compliant.

Those of skill in the art will appreciate that while the invention has been described by reference to the illustrated preferred embodiments, the illustrated embodiments can be modified in arrangement and detail without departing from the scope of the invention.

What is claimed is:

1. A suture comprising:
a hollow, flexible cover comprising a plurality of interlocked, abrasion resistant polymeric fibers; and,
a cushioning core disposed within the cover.

2. A suture according to claim 1, wherein the cushioning core is comprised of a plurality of twisted fibers.

3. A suture according to claim 1, wherein the cushioning core is polyester.

4. A suture according to claim 1, wherein the core is operable to support the cover in a first cross-sectional shape when the suture is in a first straight configuration, and to permit a cushioning deformation of the cross-sectional shape of the cover when the suture is in a second bent configuration.

5. A suture according to claim 2, wherein the plurality of fibers are twisted together to form threads and the threads are twisted together to form the core.

6. A suture according to claim 2, wherein the plurality of fibers are twisted together at between 0.5 and 2.5 turns per inch.

7. A suture according to claim 2, wherein the plurality of fibers are twisted together at about 1.5 turns per inch.

8. A suture according to claim 5, wherein the threads are twisted together at between 0.5 and 2.5 turns per inch.

9. A suture according to claim 5, wherein the threads are twisted together at about 1.5 turns per inch.

10. A suture according to claim 1, wherein the core comprises about 15% to 25% of the suture.

11. A suture according to claim 1, wherein the core comprises about 22% of the suture.

12. A suture comprising:
a hollow, flexible cover comprising a foraminous, abrasion resistant polymeric material; and,
a cushioning core disposed within the cover.

13. A suture according to claim 12, wherein the cushioning core is comprised of a plurality of twisted fibers.

14. A suture according to claim 12, wherein the cushioning core is polyester.

15. A suture according to claim 12, wherein the core is operable to support the cover in a first cross-sectional shape when the suture is in a first straight configuration, and to permit a cushioning deformation of the cross-sectional shape of the cover when the suture is in a second bent configuration.

16. A suture according to claim 13, wherein the plurality of fibers are twisted together to form threads and the threads are twisted together to form the core.

17. A suture according to claim 13, wherein the plurality of fibers are twisted together at between 0.5 and 2.5 turns per inch.

18. A suture according to claim 13, wherein the plurality of fibers are twisted together at about 1.5 turns per inch.

19. A suture according to claim 16, wherein the threads are twisted together at between 0.5 and 2.5 turns per inch.

20. A suture according to claim 16, wherein the threads are twisted together at about 1.5 turns per inch.

21. A suture according to claim 12, wherein the core comprises about 15% to 25% of the suture.

22. A suture according to claim 12, wherein the core comprises about 22% of the suture.
